(19) United States
(12) Patent Application Publication
(10) Pub. No.: US 2022/0249170 A1
(43) Pub. Date: Aug. 11, 2022

(54) SYSTEM AND METHOD FOR PROCESSING BLACK BONE MRI DATA

(71) Applicant: Surgical Theater, Inc., Los Angeles, CA (US)

(72) Inventors: MORDECHAI AVISAR, HIGHLAND HEIGHTS, OH (US); Alon Yakob Geri, Orange Village, OH (US); Whitney LAI, Emeryville, CA (US); Andrew CARLSON, Jeffersontown, KY (US)

(73) Assignee: SURGICAL THEATER, INC., Los Angeles, CA (US)

(21) Appl. No.: 17/667,261

(22) Filed: Feb. 8, 2022

Related U.S. Application Data
(60) Provisional application No. 63/147,200, filed on Feb. 8, 2021.

(51) Int. Cl. A61B 34/10; G01R 33/561; G01R 33/56; G02B 27/01; G06T 7/00; A61B 90/00

(52) U.S. Cl. CPC A61B 34/10; G01R 33/5613; G01R 33/5608; G02B 27/017; G06T 7/0012; A61B 2090/374; G06T 2207/10088; G06T 2207/30008; A61B 2090/365; A61B 2034/105; A61B 90/37

(57) ABSTRACT

A system and method for providing an imaging system utilizing black bone MRI scanning data of a particular patient. Post processing software is provided for executing on a computer system to process the Black Bone MRI dataset into a 360VR model. This model highlights bone structures of the particular patient. The post processing software first inverts the dataset and then utilizes an auto detection algorithm that detects the pixels of intensity range similar to that of bone. Additional tools such as an erase tool that removes pixels out of intensity range within the designated bounds of the area, were developed to help further clean up the model, thereby providing a model useful for planning or performing medical procedures on the patient.

### CROSS-REFERENCES TO RELATED APPLICATIONS

This application claims the benefit of U.S. provisional application Ser. No. 63/147,200 filed on Feb. 8, 2021, which is incorporated herein by reference.

### BACKGROUND

Surgical procedures may often be complex and time sensitive and vary in scope from one patient to another. For example, in the case of bone surgery, the point of repair may vary in terms or procedural requirements depending on the exact location, size, and so on. Accurate views of the bones of the patient are desirable to ensure proper treatments are provided. The accuracy and efficiency of the procedure is highly critical and detailed planning based on the patient specific local geometry and physical properties of the area on which surgery is being performed is fundamental. To achieve a new level of pre-surgery preparation, 3D CT and MRI images are being increasingly utilized. But bone imaging is particularly difficult since MRI data has proven deficient, requiring reliance on CT scans and x-rays, which can be inadequate for showing important and desirable features.

Computed tomography (CT) is the current gold standard for imaging bony pathologies in neurosurgery, spine, and orthopedics due to the short acquisition time, superior bone depiction for sutures and fractures, lower cost and availability. While standard magnetic resonance imaging (MRI) is a non-ionizing imaging method, its long acquisition time and poor resolution of bone make it a suboptimal candidate for imaging bony pathologies. The low resolution of bone on standard MRI results from the low proton content in hard tissues These challenges are a result of the low proton content and short transverse relaxation times of hard tissues.

One of the biggest concerns is ionization radiation exposure particularly in children. Often, due to the risk of ionizing radiation, post-op imaging is not obtained, limiting post-op VR interactions. Post-op imaging of bony tissue could increase the utilization of ST products for patient engagement. A means of utilizing the benefits of MRI scanning for bone tissue is desirable.

### SUMMARY

Provided herein is a method for processing a black bone MRI dataset into a virtual model, comprising the steps of: performing a black bone MRI on a bone and tissue of a particular patient; obtaining a black bone dataset of the patient from the black bone MRI; processing the black bone dataset; and generating a dynamic virtual model of the bone and tissue of the patient from the processed black bone dataset.

Also provided is method for processing a black bone MRI dataset into a virtual model, comprising the steps of: performing a black bone MRI on a bone and tissue of a particular patient, wherein said black bone MRI utilizes an echo sequence with a low flip angle gradient providing high contrast between bone and tissue; obtaining a black bone dataset of the patient from the black bone MRI; processing the black bone dataset utilizing an auto detection algorithm that detects the pixels of intensity range similar to that of bone from the black bone dataset; and generating a dynamic virtual model of the bone and tissue of the patient from the processed black bone dataset, wherein said virtual model is configured to highlight bone structures of the patient.

Further provided is a method for processing a black bone MRI dataset into a virtual model, comprising the steps of: performing a black bone MRI on a bone and tissue of a particular patient, wherein said black bone MRI utilizes an echo sequence with a low flip angle gradient providing high contrast between bone and tissue, and wherein black bone MRI utilizes an echo time (TE) sequence of 4.2 ms. and a repetition time of 8.6 ms. at a 5 degree flip angle; obtaining a black bone dataset of the patient from the black bone MRI; processing the black bone dataset by inverting the black bone dataset and utilizing an auto detection algorithm that detects the pixels of intensity range similar to that of bone from the black bone dataset; and generating a dynamic virtual model of the bone and tissue of the patient from the processed black bone dataset, wherein said virtual model is a 3D 360VR model configured to highlight bone structures of the patient.

Still further provided is a system including a computer system for performing any of the above methods.

Also provided are additional example embodiments, some, but not all of which, are described hereinbelow in more detail

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for augmented reality simulations using Black Bone MRI Data.

FIG. 2 illustrates example images utilizing Black Bone MRI Data prior to processing.

FIG. 3 illustrates example images utilizing Black Bone MRI Data subsequent to processing.

FIG. 4 shows a table of example settings for the black bone MRI imaging process;

FIG. 5 illustrates an example model image for augmented reality simulations.

FIG. 6 illustrates an example method for processing black bone MRI data.

FIG. 7 illustrates an example computer implementing the example system of FIG. 1.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3 Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. Pat. No. 8,311,791, incorporated in this application by reference, has been developed to convert static CT and MRI medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional two-dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time by an image generator using a SNAP model built from the patient's own data set of medical images including CT, MRI, DTI etc., and is patient specific, such as a SNAP computer previously described in U.S. Pat. No. 8,311,791, incorporated herein by reference. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved and can be appreciated using the MD6DM.

The algorithm of the MD6DM rendered by the image generator takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

Described herein is an imaging system, leveraging an image generator and a MD6DM model, for creating a synchronized augmented reality view of a subject utilizing black bone MRI data for creating the models. In particular, the imaging system enables augmenting and overlaying the MD6DM model over top of a corresponding physical model or real-time patient images. Moreover, the imaging system anchors the MD6DM model to the physical model or patient and synchronizes the two, such that a new image is created and overplayed over top of the physical model according to movement around the model. This is accomplished by streaming the image generator directly to an HMD, tracking a position and location of the HMD, and adjusting the image generator based on the tracked movement. Thus, a dependency is created between the virtual model and the physical model.

By creating such a dependency and tying or anchoring a virtual model to a physical model or patient, and then adjusting an image overplayed on top of the physical model based on movement with respect to the physical model, a HMD is able to receive a synchronized augmented reality view of the physical model regardless of where a user of the HMD is positioned with respect to the physical model, thus offering the user an improved perspective of the physical model. As a result of anchoring the virtual model to the physical model, the visual model is not separated from the physical model. In other words, if a user of the HMD turns his head and looks away from the physical model, the user will no longer see the virtual model either. Only when the user returns focus to the physical model will the user again see the virtual model, overlayed and synchronized as appropriate. Thus, a user may be presented with the augmented view of a main physical object while still providing the user with the freedom and flexibility to maneuver and interact with secondary physical objects within proximity of the main physical object without interfering with the user's view of or interaction with the secondary objects.

It should be appreciated that although reference is made to anchoring or tying a virtual model to a physical model, the virtual model may be anchored to a physical location, rather than to a physical object, and it is understood that the physical object's position does not move during the augmented reality viewing of the physical object.

It should be appreciated that although the examples described herein may refer in general to medical applications and specifically to virtual models or images of a patient's anatomy augmented and synchronized with a corresponding patient's physical body for the purpose of performing spine surgery, the imaging system may similarly be used to synchronize and augment a virtual model or image of any virtual object with a corresponding physical object.

The approach disclosed in this application involves utilizing black bone MRI imaging to build the desired virtual models for use in generating complex dynamic models and augmented reality views for planning and executing medical procedures on particular patients. This approach solves the problem of rendering bony tissue from MRI scans using Black Bone MRI. This Black Bone approach offers an alternative imaging technique to CT imaging that could reduce radiation exposure and additional important MRI dataset.

Black Bone MRI, a unique MRI acquisition technique, has shown promise in recent studies as a reliable alternative to head CT for imaging bone. Black Bone MRI utilizes an echo sequence with low flip angle gradient that minimizes surrounding soft tissue to provide high contrast between bone and tissue. This imaging technique involves a short echo time (TE) sequence of 4.2 ms/8.6 repetition time (8.6 ms) at 5 degree flip angle with 1.5 or 3.0 T magnet.

The echo time (TE) refers to the time between the application of the radiofrequency excitation pulse and the peak of the signal induced in the coil. It is measured in milliseconds. The amount of T2 relaxation is controlled by the TE.

The “Black Bone MRI” protocol sequence from the University of Oxford publication was adapted for use in this disclosed process. Using this protocol, a post processing software was developed to process the Black Bone MRI dataset into a 360VR model. This model highlights bone structures. The post processing software first inverts the dataset and then utilizes an auto detection algorithm that detects the pixels of intensity range similar to that of bone. Additional tools such as an erase tool that removes pixels out of intensity range within the designated bounds of the area, were developed to help further clean up the model.

The new post-processing algorithm of a “Black Bone” MRI sequence (bbMRI) is a radiation-sparing tool for visualizing and diagnosing bony pathologies with an identical sensitivity and specificity as traditional head CT.

The MRI guidance approach relies on data input for accuracy of bone approximation. Since the scans acquired use a imaging sequence that produces high contrast bone and tissue, our software does not require additional data or machine learning sequence on the entire dataset.

It should be appreciated that, although references may be made herein to building a virtual model using the processed black bone MRI imaging, the processed Black Bone MRI may, in one example, also be used independently of any specific virtual or augmented reality application or without performing any virtual modeling.

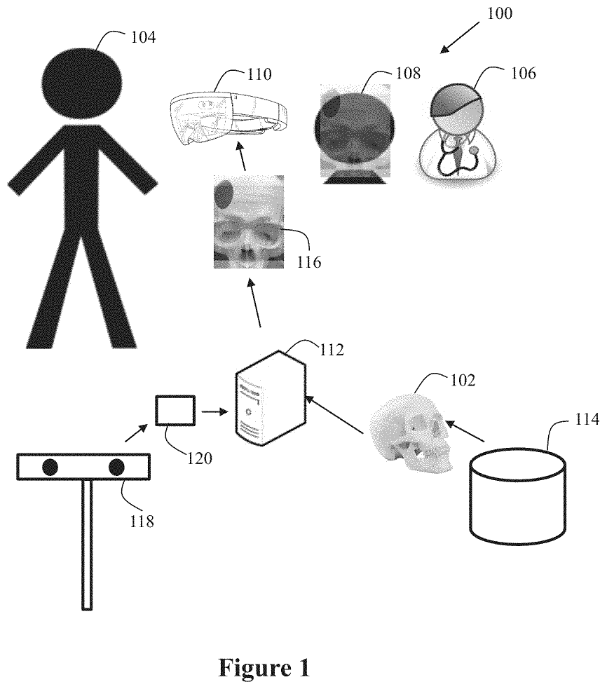

FIG. 1 illustrates a system 100 for augmenting and synchronizing a virtual model 102 with a physical model 104. In particular, the system 100 enables a user 106, such as a physician, to view an augmented realty view 108 of the physical model 104 from any perspective of the physical model 104. In other words, the user 106 may walk around the physical model 104 and view the physical model 104 from any side, angle, or perspective, and to have the synchronized corresponding view of the virtual model 102 overlayed on top of the physical model 104 in order to form the augmented realty view 108. And, if the user 106 turns away from the physical model 104 such that the physical model 104 is no longer within a current view or line of sight, the virtual model 102 similarly is also eliminated from the current view or line of sight.

The virtual model(s) 102 may provide additional biological features for adding to the physical model 104, such as by providing virtual models of internal organs and/or musculature to a physical model of a skeleton, for example. Either or both the virtual model(s) 102 and the physical model 104 may be generic models or models based on the physical biological characteristic of an actual patient as determined by various imaging scanning techniques. The virtual model(s) 102 might alternatively, or additionally, include models of various tools, implants, or other physical entities.

The system 100 includes an augmented reality head mounted display (“HMD”) 110 for providing the user 106 with augmented realty view 108 including a live real life visual of the physical model 104 in combination with additionally integrated content, such as the virtual model 102. For example, the system 100 includes an AR synchronization and image processing computer 112 for accessing the black bone MRI data and images 120 from an MRI imaging system 118 for processing the data, retrieving a virtual model 102 such as a SNAP model, from a virtual model database 114, for rendering a virtual image 116 from the virtual model 102, and for providing the virtual image 116 to the HMD 110. In one example, the AR synchronization computer 112 includes an image generator (not shown) for rendering the virtual image 116 from the virtual model 102. In another example, the image generator is specific to a virtual model 102 and is included with the virtual model 102 retrieved from the virtual model database 114.

It should be appreciated that although the AR synchronization computer 112 is depicted as being external to the HMD 110, in one example, the AR synchronization computer 112 may be incorporated into the HMD 110. This provides for a single integrated solution for receiving and processing a virtual model 102 so that the HMD 110 may provide the user with the augmented reality view 108 as described. In such an example, the virtual model 102, or image generator for the virtual model 102, is streamed directly to the HMD 110.

The AR synchronization computer 112, in combination with the MD 110, is configured to tie or anchor the virtual model 102 to the physical model 104 and to synchronize the virtual model 102 with and overlay it on top of the live real life visual of the physical model 104 in order to create the augmented realty view 108 of the physical model 104 via the MD 110. In order to facilitate generating bone images, the AR synchronization computer 112 is configured to communicate with the black bone MRI system 118. In particular, the AR synchronization computer 112 is configured to receive black bone imaging data and images 120 from the MRI system 118 and to generate model images 116 using the black bone imaging data and images 120 and other stored model images 102. The AR synchronization computer 112 can then generate the 3D models from the imaging data 120 and the virtual models 102, tying them to the physical model/patient 104. Once anchored, the AR synchronization computer 112 is able to generate the appropriate virtual image 116 depending on tracked movement of the HMD 110 via the navigation system 118, and the data MRI 120.

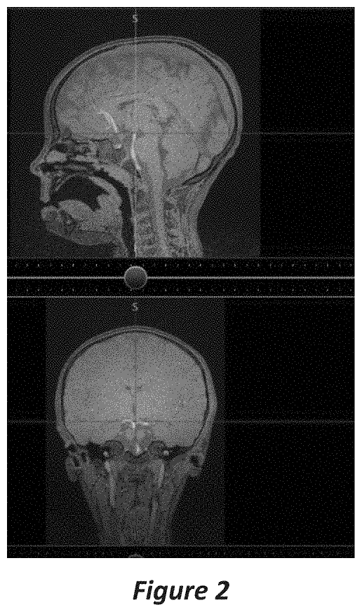

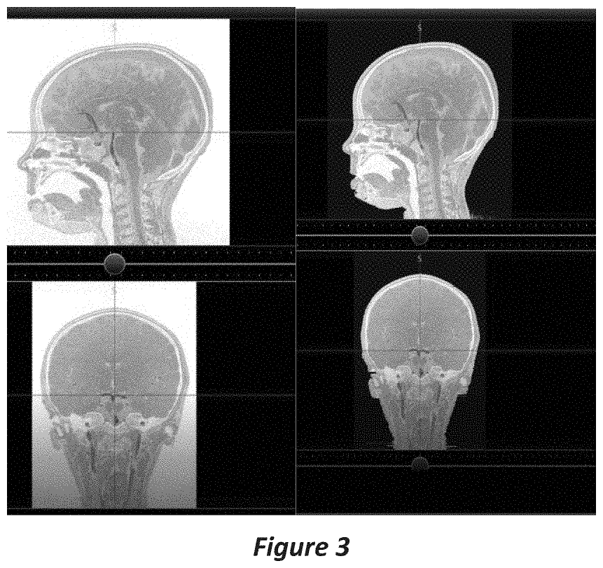

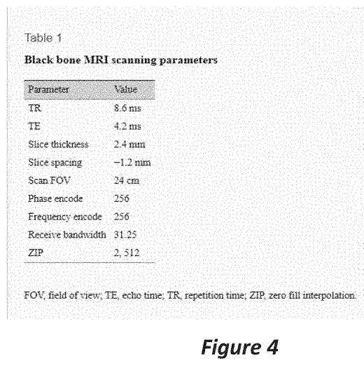

FIG. 2 illustrates example images obtained from black bone MRI imaging prior to processing. FIG. 3 shows these images subsequent to processing. FIG. 4 shows a table with the scanning parameters used to generate the example images.

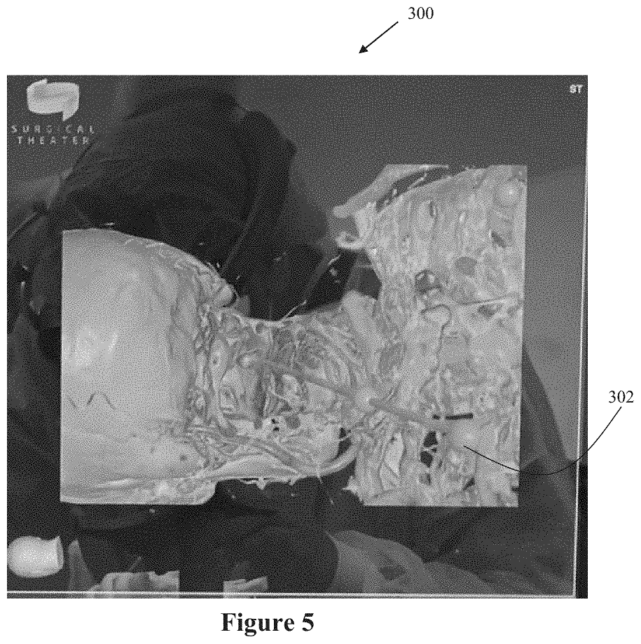

FIG. 5 illustrates an example model image 300 and virtual tool 302 for augmented reality simulations which can be used to incorporate and overlay a processed Black Bone MRI image.

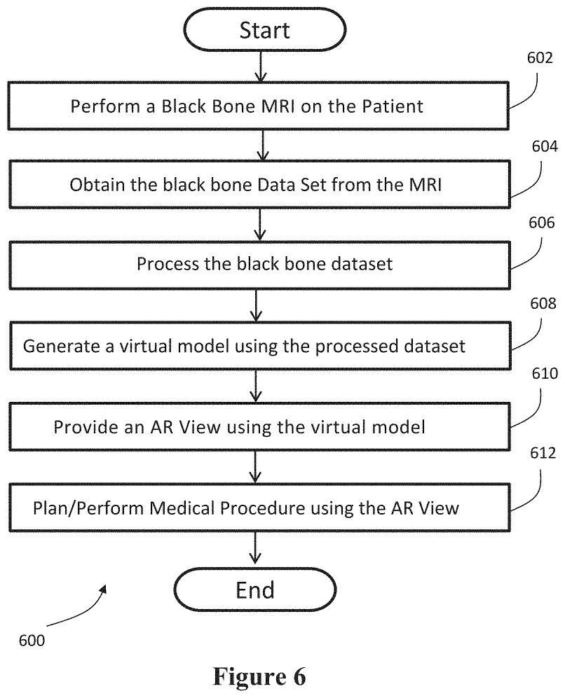

FIG. 6 provides an example process 600 for generating Augmented Reality (AR) views utilizing the black bone MRI data for use in planning or performing medical procedures for a particular patient. The black bone MRI is performed on the patient 602. A black bone data set is obtained from the MRI 604. This dataset is then processed 606, for use in generating a virtual model 608. The virtual model can then be used to provide an AR model 610 that can be used to plan or perform the medical procedure 612.

Various types of processing can be performed on the black bone MRI data, including inverting the black bone dataset, utilizing an auto detection algorithm that detects the pixels of intensity range similar to that of bone from the inverted black bone dataset, and/or utilizing an echo sequence with a low flip angle gradient providing high contrast between bone and tissue. The processing can include removing pixels out of intensity range within designated bounds of an area using an erase tool, for example.

In a preferred embodiment, the black bone MRI utilizes an echo time (TE) sequence of 4.2 ms. and a repetition time of 8.6 ms. at a 5 degree flip angle using a 1.5 T or 3.0 T magnet. Also, the MRI can utilize an echo sequence with a low flip angle gradient providing high contrast between bone and tissue.

In a preferred embodiment, the virtual model is a 3D 360VR model configured to highlight bone structures of the patient. An example use of the model is for visualizing and diagnosing bony pathologies.

In one example, as illustrated in FIG. 5, a virtual model 300 is provided showing images based on scanning data including a tool image 302. For example, data such as 2D or 3D models and renderings of various tools may be retrieved from a database.

As can be appreciated, the system described herein provides numerous benefits to a user or a physician. For example, using the augmented reality system for bone surgery or implant placement, or for any other surgical procedure, allows the surgeon to better prepare for the surgery and perform surgery in a safer manner. This is made possible because of the unique and novel view presented to the surgeon which allows the surgeon to view a combination of bone and anatomy including soft tissue, nerves, spine, blood vessels, lungs, etc. and to view an anatomy even if it is obscured by other tissue.

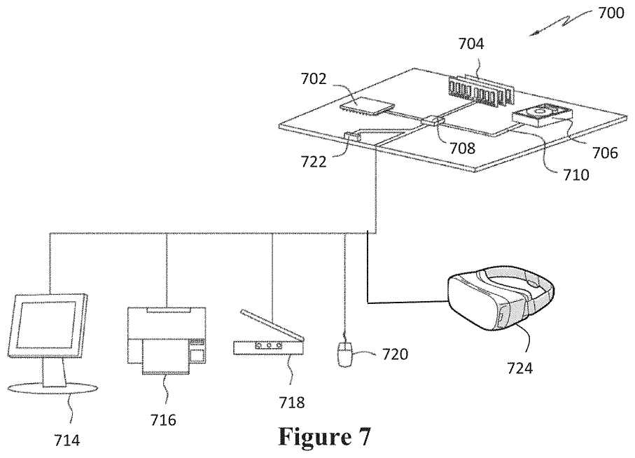

FIG. 7 is a schematic diagram of an example computer for implementing the AR synchronization computer 112 of FIG. 1. The example computer 700 is intended to represent various forms of digital computers, including laptops, desktops, handheld computers, tablet computers, smartphones, servers, and other similar types of computing devices. Computer 700 includes a processor 702, memory 704, a storage device 706, and a communication port 708, operably connected by an interface 710 via a bus 712.

Processor 702 processes instructions, via memory 704, for execution within computer 600. In an example embodiment, multiple processors along with multiple memories may be used.

Memory 704 may be volatile memory or non-volatile memory. Memory 704 may be a computer-readable medium, such as a magnetic disk or optical disk. Storage device 706 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 704 or storage device 706.

Computer 700 can be coupled to one or more input and output devices such as a display 714, a printer 716, a scanner 718, a mouse 720, and a HMD 724.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. A method for processing a black bone MRI dataset into a virtual model, comprising the steps of:
performing a black bone MRI on a bone and tissue of a particular patient;
obtaining a black bone dataset of the patient from the black bone MRI;
processing the black bone dataset; and
generating a dynamic virtual model of the bone and tissue of the patient from the processed black bone dataset.

2. The method of claim 1, wherein said processing includes inverting the black bone dataset.

3. The method of claim 2, wherein said processing includes utilizing an auto detection algorithm that detects the pixels of intensity range similar to that of bone from the inverted black bone dataset.

4. The method of claim 3, wherein said black bone MRI utilizes an echo sequence with a low flip angle gradient providing high contrast between bone and tissue.

5. The method of claim 4, wherein said black bone MRI utilizes an echo time (TE) sequence of 4.2 ms. and a repetition time of 8.6 ms. at a 5 degree flip angle using a 1.5 T or 3.0 T magnet.

6. The method of claim 1, wherein said black bone MRI utilizes an echo sequence with a low flip angle gradient providing high contrast between bone and tissue.

7. The method of claim 6, wherein said black bone MRI utilizes an echo time (TE) sequence of 4.2 ms. and a repetition time of 8.6 ms. at a 5 degree flip angle using a 1.5 T or 3.0 T magnet.

8. The method of claim 1, wherein said processing includes utilizing an auto detection algorithm that detects the pixels of intensity range similar to that of bone from the black bone dataset.

9. The method of claim 1, wherein said black bone MRI utilizes an echo time (TE) sequence of 4.2 ms. and a repetition time of 8.6 ms. at a 5 degree flip angle using a 1.5 T or 3.0 T magnet.

10. The method of claim 1, wherein said virtual model is a 3D 360VR model configured to highlight bone structures of the patient.

11. The method of claim 10, further comprising the step of using said virtual model for visualizing and diagnosing bony pathologies.

12. The method of claim 10, further comprising the step of removing pixels out of intensity range within designated bounds of an area using an erase tool.

13. A system for implementing the method of claim 1.

14. A method for processing a black bone MRI dataset into a virtual model, comprising the steps of:
performing a black bone MRI on a bone and tissue of a particular patient, wherein said black bone MRI utilizes an echo sequence with a low flip angle gradient providing high contrast between bone and tissue;
obtaining a black bone dataset of the patient from the black bone MRI;
processing the black bone dataset utilizing an auto detection algorithm that detects the pixels of intensity range similar to that of bone from the black bone dataset; and
generating a dynamic virtual model of the bone and tissue of the patient from the processed black bone dataset, wherein said virtual model is configured to highlight bone structures of the patient.

15. The method of claim 14, wherein said black bone MRI utilizes an echo time (TE) sequence of 4.2 ms. and a repetition time of 8.6 ms. at a 5 degree flip angle using a 1.5 T or 3.0 T magnet.

16. The method of claim 14, further comprising the step of removing pixels out of intensity range within designated bounds of an area using an erase tool.

17. The method of claim 14, further comprising the step of using said virtual model for visualizing and diagnosing bony pathologies.

18. A system for implementing the method of claim 14.

19. A method for processing a black bone MRI dataset into a virtual model, comprising the steps of:
performing a black bone MRI on a bone and tissue of a particular patient, wherein
said black bone MRI utilizes an echo sequence with a low flip angle gradient providing high contrast between bone and tissue, and wherein
said black bone MRI utilizes an echo time (TE) sequence of 4.2 ms. and a repetition time of 8.6 ms. at a 5 degree flip angle;
obtaining a black bone dataset of the patient from the black bone MRI;
processing the black bone dataset by inverting the black bone dataset and utilizing an auto detection algorithm that detects the pixels of intensity range similar to that of bone from the black bone dataset; and
generating a dynamic virtual model of the bone and tissue of the patient from the processed black bone dataset, wherein said virtual model is a 3D 360VR model configured to highlight bone structures of the patient.

20. The method of claim 19, further comprising the step of using said virtual model for visualizing and diagnosing bony pathologies.
